(19) United States
(12) Patent Application Publication
(10) Pub. No.: US 2003/0001024 A1
(43) Pub. Date: Jan. 2, 2003

(54) Liquid delivery devices

(76) Inventor: Brian Parry Slade, Ashford (GB)

Correspondence Address: CESARI AND MCKENNA, LLP, 88 BLACK FALCON AVENUE, BOSTON, MA, 02210, US

(21) Appl. No.: 10/192,202

(22) Filed: Jul. 10, 2002

Related U.S. Application Data
(63) Continuation of application No. 09/704,095, filed on Nov. 1, 2000, now abandoned.

(30) Foreign Application Priority Data
Nov. 2, 1999 (EP) 99308706.3

(51) Int. Cl. A61L 9/04

(52) U.S. Cl. 239/42

(57) ABSTRACT

A liquid delivery device is particularly suited for dosing a toilet bowl with cleaning agents. Liquid from a reservoir flows through an inner sleeve of a connector into a cup until the liquid level reaches the mouth of the inner sleeve. Web is perforated so that the space in cup is open to atmosphere. Liquid is drawn from the cup by a capillary formed between the wall of cup and the connector and delivered to a porous pad. Flushwater runs over pad and supporting plate to disperse the liquid into a toilet bowl.

### INTRODUCTION AND BACKGROUND

This invention relates to devices for delivering a liquid product. The invention has particular application in areas where long term and controlled release of a liquid product is required, the product being dispensed or dispersed by evaporation, such as in an air freshener or insecticide, or into a flow of other dispersing liquid such as in toilet systems, particularly systems which have an intermittent flow of the dispersing liquid. The invention is particularly concerned with delivering a liquid product such as a perfume, surfactant, bleach or disinfectant, particularly in the form of a solution, dispersion or suspension, and for delivering it to a toilet bowl under the action of water used to flush the toilet bowl.

It has been known for a long time to provide so-called toilet automatics in the form of a solid or semi-solid product, a ‘rim block’, to be mounted within the inner rim of a water closet bowl where the flushing water will wash over the product and so dissolve or erode it to release active constituents into the water flow. Blocks may also be placed on top of the cistern, in Japanese style systems where water from a tap flows over the block and then into the cistern, and also may be placed within the cistern below the water level, where they slowly release constituents into the water.

More recently, it has been proposed to use a liquid toilet freshening product in a similar manner, a so-called liquid rim product. For example, EP-A-0538957 describes a device that can be mounted on the inner rim of a water closet bowl to dose a liquid freshening product into the flushing water. In this device, the liquid product is dosed into the water flow from a porous substrate, a delivery plate, which is disposed in the path of the flushing water. The porous substrate is supplied with the liquid product from a container disposed above the substrate, a mouth at the bottom of the reservoir opening onto the upper surface of the substrate. Although this arrangement is simple in construction, it suffers from the drawback that the volume of liquid product that flows to the substrate between flushes is not consistent over the life time of the product, which is typically intended to be 3 to 4 weeks. Dosing seems to depend at least in part, on the head of liquid in the container, since this directly influences the rate of flow from the container onto the surface of the substrate. The container is sealed above the opening, and so a reduced pressure is created above the liquid as it flows onto the substrate. The result is an inconsistency in the dose of liquid product into the toilet bowl over time.

EP-A-07853 15 describes a development of the device discussed above. The same basic principle of delivering a liquid product into a flow of water from a porous substrate is employed. However, liquid product from a container is deposited onto the upper surface of the substrate via a regulating channel. The liquid is metered into the channel through an orifice and a separate air opening to the interior of the container is provided. The sizes of the metering orifice and the air opening are strictly regulated to the viscosity of the liquid being dosed. This is described as having the effect of providing a substantially constant ‘head’ of the liquid above the substrate, independent of the level in the container, although the height of liquid in the container necessarily reduces over time. Although this arrangement provides a more consistent flow rate of liquid product to the absorbent substrate, inconsistent delivery to the flushing water can still result, dependent at least in part on the duration of the periods between flushes. This is thought to be due to the reliance of this device on coagulation of the liquid product to stem its flow onto the substrate, a mechanism which is very dependent on the environment in which the device is operated. It is also thought that the head of liquid bearing down on the substrate can lead to ‘supersaturation’ of the substrate, so it becomes over loaded with product.

WO 99/66139 and WO 99/66140 describe numerous variations of the liquid rim product, including different styles of delivery plate in place of the porous plate of EP-A-0 538 957, while WO 00/42261 describes yet another product using a grooved plate.

All of the systems still use the same basic idea of delivering liquid directly from the container's mouth onto the delivery plate.

Also well known are perfume delivery systems in which a container of liquid is open at its top, as described in EP-A-669137. A wick in the liquid feeds the liquid up to a felt or other porous pad to create a larger surface area of liquid, from which the perfume can evaporate into the ambient atmosphere.

### SUMMARY OF THE INVENTION

It is one object of the present invention to provide a device which can introduce a substantially consistent dose of a liquid product to the delivery plate as the container empties over time

One aspect of the present invention provides a liquid delivery device comprising a container for the liquid, the container having an outlet at a lower end thereof, and a cup surrounding a mouth of the container outlet, the cup being open to the atmosphere, whereby in use liquid flows from the container into the cup until the container mouth is covered by liquid in the cup, a reduced pressure being created above the liquid in the container and preventing further flow of liquid into the cup, wherein dispersal means is provided to disperse the liquid from the cup.

Preferably the dispersal means comprises a capillary based arrangement for transporting liquid from the cup to a delivery plate. Liquid from the delivery plate may then evaporate into atmosphere, or be dispersed by a flow of other liquid over the plate, for example.

The capillary may be in the form of a porous member or pliable wick such as a cellulosic or polyester wick used in air freshener devices, but preferably an arrangement of elongate capillary channels is provided.

The cup will contain a relatively constant head of liquid. As liquid is dispersed from the cup, the liquid level in the cup falls below the level of the container mouth, at which point air can enter the container and liquid from the container flows down into the cup to replenish it.

The cup may be formed as part of the container outlet. Thus another aspect of the invention provides a liquid delivery device comprising a container for the liquid, the container having an outlet at a lower end thereof, when the container is positioned for use, and a mouth of the container outlet extending upwardly, whereby the outlet forms a reservoir of liquid at the lower end of the container, and dispersal means is provided for dispersing the liquid from the outlet. Preferably the dispersal means is a capillary channel or channels for feeding the liquid to a substrate such as a plate or porous mass providing an enlarged, exposed area of liquid.

In yet another aspect the invention provides a device for delivering a liquid product into a receiver in conjunction with a liquid flow into said receiver, the device comprising a substrate, which in use is exposed to said liquid flow, a cup disposed below the substrate for receiving liquid product from a container and having a free liquid level open to atmosphere, and means for transporting the liquid product from the cup upwardly to the substrate.

It has been found that by supplying the liquid product in this manner, the product dose can be maintained substantially consistent, independent of factors such as the level of liquid product in the container and the time between flushes. The take-up of liquid product by the substrate is due substantially to the head of liquid in the cup and properties of the dispersal means. In particular, the supply of product to the substrate can be stopped or reduced to a negligible amount, when the substrate becomes saturated.

The dispersal means may comprise at least one passage extending from the cup at or near its base, to the underside of the substrate. The liquid product in the cup then provides a hydrostatic head which serves to drive the liquid product at least part way up the passage.

The level of liquid in the cup, and hence the head it generates, may be controlled such that the product is urged upwardly through the passage up to, but preferably no higher than, its opening to the substrate.

Desirably, the upward transport of the liquid product from the cup to the substrate relies at least in part on capillary action. For example, the dispersal means may include one or more capillary passages extending upwardly from the cup towards the base of the substrate, e.g. as discrete passages formed in the device or in the form of a wick extending upwardly from the cup. By relying on capillary action in this way, it has been found that the flow of liquid to the substrate is more surely stopped once the substrate becomes saturated.

Accordingly, in a more particular aspect of the invention, there is provided a device for delivering a liquid product into a receiver in conjunction with a liquid flow into said receiver, the device comprising a substrate, which in use is exposed to said liquid flow, a cup disposed below the substrate for receiving liquid product from a reservoir and having a free liquid level open to atmosphere, and means for transporting the liquid product from the cup upwardly to the substrate, said transport means comprising one or more capillary passages opening onto the substrate.

However, to rely solely on capillary action to lift the product to the substrate may be impractical, particularly if it is necessary to lift the liquid through anything other than a small height. This is because the small cross-sectional passage required to give greater capillary lift may reduce the flow rate of the product through the passage to such an extent that the porous substrate is not replenished fully between flushes. It is therefore particularly preferred to use a combination of hydrostatic pressure and capillary action to transport the liquid product to the substrate. In this way, the capillary rise required can be kept to a minimum, since it is only necessary to rely on capillary forces to lift the liquid to the substrate from the level it is taken to by the hydrostatic forces acting on it.

Thus, in a preferred form of the invention, one or more passages are provided which extend from the cup, at or near its base, to the substrate, at least an upper portion of the or each passage having a cross-sectional area sufficiently small to create the desired capillary action. The actual cross-sectional area of this upper portion can be selected based on the properties of the liquid product and the capillary rise necessary to transport the liquid to the substrate.

### BRIEF DESCRIPTION OF THE DRAWINGS

The invention will be described in more detail, by way of example, with reference to the accompanying drawings, in which:

FIG. 1 is a schematic side elevation of a device forming an embodiment of the invention, including a liquid container and showing its mounting position relative to the rim of a water closet bowl;

FIG. 2 is a front sectional view of the device of FIG. 1, with the container separated;

FIG. 3 is a plan view of the device of FIG. 1 with the container removed;

FIG. 4 is a cross-section through a detail of a second embodiment of a liquid dispensing device in accordance with the invention;

FIG. 5 is a side view of an insert to be placed in a cup of the device of FIG. 4;

FIG. 6 is a cross-section on a diameter of the insert of FIG. 5;

FIG. 7 is a cross-section on line VII-VII of FIG. 4;

FIG. 8 is a cross-section through a third embodiment of a liquid delivery device in accordance with the invention, and

FIG. 9 illustrates yet another embodiment of the invention.

### DESCRIPTION OF THE PREFERRED EMBODIMENTS

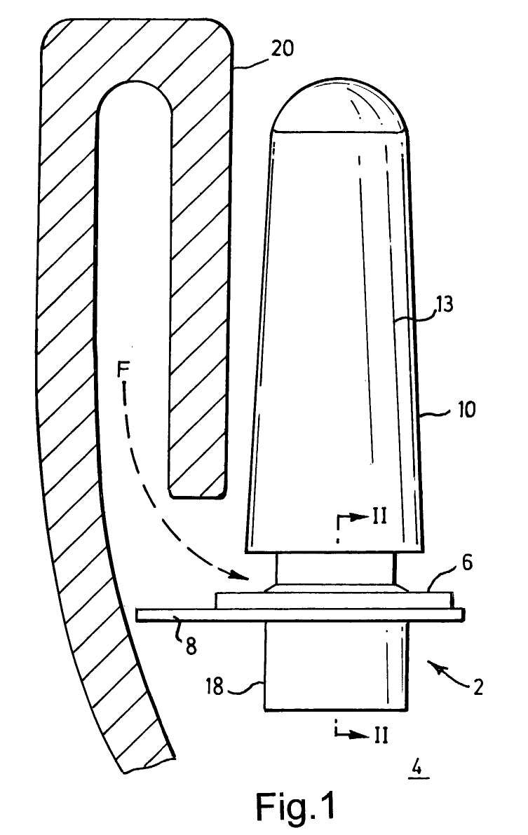

The device 2 of FIGS. 1 to 3 is adapted for delivery a liquid product, such as a cleansing and/or deodorising product into the bowl 4 of a water closet, in conjunction with the flow of water F generated when the water closet is flushed. The device comprises a porous substrate 6 which is supported on a generally horizontal platform 8 to form a delivery plate in the path of the flushing water F. A container 10, mounted on the device 2 above the platform 8, serves as a reservoir for the liquid product 13 which is fed under the influence of gravity to a cup 18 disposed below the platform 8. The liquid product 13 is transported upwardly from the cup 18 to dose the substrate 6. Although not shown in FIGS. 1 to 3, the device also includes a strap via which it can be suspended from the rim of the water closet bowl (much in the same way as a conventional ‘rim block’), and may be surrounded by a cage-like structure to offer some protection to the substrate 6.

Looking at the device in more detail, it has a base 16 of polypropylene, for example, which includes the circular cup 18 and a rectangular platform 8, which surrounds and extends outwardly from the mouth of the cup 18. The cup 18 is set centrally in the platform 8 in its lateral dimension, but is offset towards the front of the platform 8 in order that, as seen in FIG. 1, a substantial part of the rear of the platform 8 can protrude below the rim 20 of the water closet bowl 4 to deflect a greater volume of flushing water over the porous substrate 6.

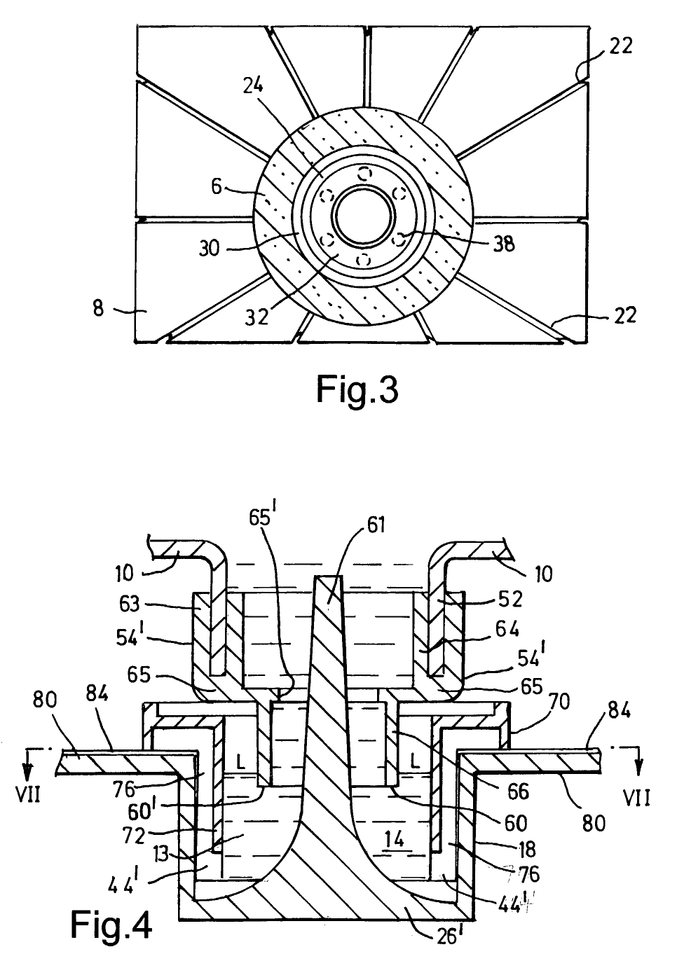

As seen most clearly in FIG. 3, a plurality of generally radially extending channels 22 are formed in the upper surface of the platform 8, which serve to encourage the flow of flushing water outwardly from the porous substrate 6, to carry the liquid product into the water closet bowl 4 and to quickly drain excess flushing water away from the substrate 6 when the flushing flow ceases.

The substrate 6 itself is annular in configuration and is supported on the platform 8 so as to surround and slightly overlap the open mouth of the cup 18.

Any of a number of different materials may be used for the porous substrate, the particular form of material being selected based on the application to which it will be put, to provide the desired absorption and flushing out of the liquid product whilst not retaining too great a volume of the flowing liquid into which it is dosed. For instance, where the flowing liquid is water, as in the embodiment described, a material with hydrophobic properties is preferred. One satisfactory material for use in a flow of flushing water has been found to be a high density polyethylene (HDPE) having a pore size of about 45-90 μm and a pore volume of about 40-50%, available from Porex Technologies.

A cylindrical, tubular insert 24 is received snugly in the cup 18, the lower end of this insert 24 being butted against the base 26 of the cup 18 and its upper end 28 protruding from the cup 18. A flange 30 protrudes radially outwardly from the wall of the insert 24, just above the mouth of the cup 18, the porous substrate 6 being trapped between this flange 30 and the platform 8 to secure it in place.

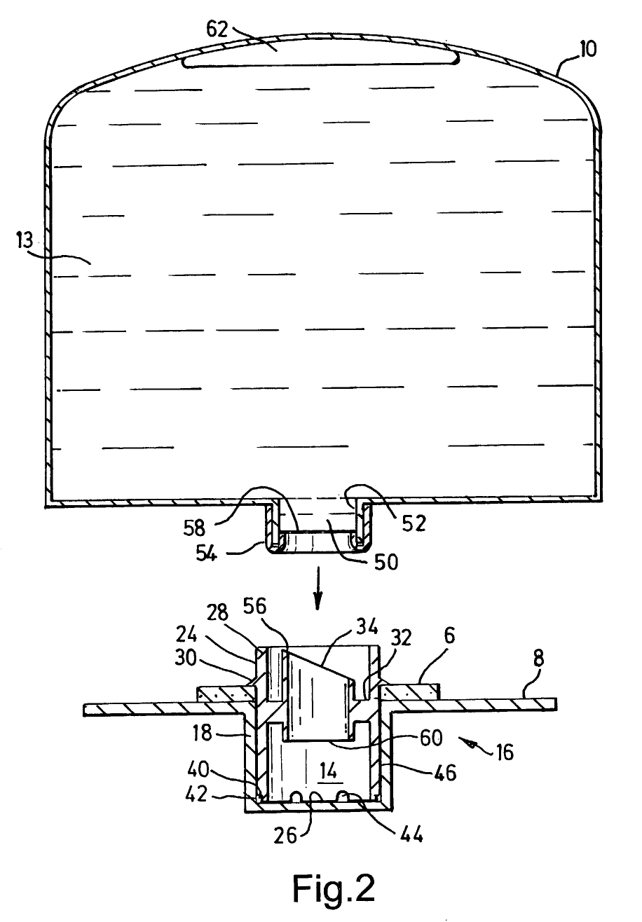

Supported concentrically within the insert 24 by a horizontal partition 32 is a delivery tube 34 which forms an outlet of the container 10, providing a conduit from above the partition 32 to below the partition for a supply of liquid 13 from the container 10 to the inner volume 14 of the cup 18, which as can be seen in FIG. 2 is circumscribed by the base 26 of the cup and the wall of the tubular insert 24. In the partition 32, surrounding the delivery tube 34, there are a plurality of through apertures 38 (FIG. 3) by which the volume 14 is vented to atmosphere. The delivery tube 34 terminates in the volume 14 at a position below these apertures or air vents 38.

An annular shoulder 40 is formed in the outside of the wall of the tubular insert 24 at its base, so that when the insert 24 is located in the cup 18 an annular gallery 42 is formed around the circumference of the cup 18. Of course, the shoulder can be supplemented or substituted by a circumferential channel formed in the side wall of the cup to provide this gallery. The gallery 42 is in fluid communication with the inside of the tubular insert 24 by way of a series of openings 44 through the wall of the insert, in this example in the form of notches 44 around its base. Liquid product can therefore pass through these openings 44 from the from the well 14 into the circumferential gallery 42.

Extending upwardly from the gallery 42, between the outer surface of the tubular insert 24 and the inner surface of the cup 18 are a series of passages 46, spaced circumferentially around the cup 18. These passages 46 open at their upper ends, at the mouth of the cup 18, to the underside of the porous substrate 6. The passages 46 have a cross-sectional area which, at least in upper portions thereof adjacent the mouth of the cup, is sufficiently small to create a capillary rise of the liquid product within the passage 46. For reasons that will become apparent below, the capillary portion of each passage 46 extends down at least as far as the level to which the delivery tube 34 protrudes into the volume 14.

The passages 46 may be formed, for example, by generally vertically extending channels formed in one or both of the opposed surfaces of the cup 18 and insert 24, or helical channels if a longer length of passage is desired. Alternatively, rather than providing a series of discrete passages, it would be possible simply to rely on a predetermined annular spacing between the cup 18 and insert 28. Suitable projections could, for example, be formed on the one or both of the opposed faces of the cup and insert to maintain the desired spacing all around.

The container 10 is rigid, or at least semi-rigid, so that it does not collapse as it empties. It is separable from the tubular insert 28 in order that it can be easily refilled, or more typically replaced; that is to say ‘refill’ containers can be provided much in the same way as replacement ‘rim blocks’ are provided for the ‘toilet fresheners’ referred to in the introduction. The overall shape of the container 10 is largely immaterial, but a slim configuration is preferred so as not to project too far across the water closet bowl 4. If desired, the walls of the container 10 can be transparent, so that the amount of liquid product remaining can be easily ascertained. The product may be coloured to make this determination of level even easier.

As illustrated in FIG. 2, the container 10 has an opening 50 in its bottom wall with a downwardly depending neck 52. The opening 50 of the container is, as seen in FIG. 2, initially closed by a seal 54 secured over the neck 52. To mount the container 10 on the delivery device 2, the neck 52 of the container 10 is pushed down onto the upper end 56 of the delivery tube 34. A recessed, circular central portion 58 of the seal 54 is contacted and broken by the upper end 56 of the delivery tube 34, which is formed at an angle, so that the seal 54 is broken with a shearing action from one side of the neck 52 to the other. The outside diameter of the delivery tube 34 and the inside diameter of the neck 52, with the remaining portion of the seal 54 thereon, are selected to provide an effective seal between these parts. This type of construction is well known in liquid rim devices. Thus, the only path by which fluid can escape from the container 10 is through the delivery tube 34 into the well 14. When the container 10 is mounted on the device a clearance is maintained between the outer circumference of the neck 52 and the wall of the insert 24 in order that the air vents 38 in the partition 32 are not blocked.

The device can be used for delivery of a variety of liquid products into a liquid flow. Typically, for the exemplary application described-cleansing and deodorising a water closet bowl-the product will include both surfactant and perfume components. The rheological behaviour of the material, in particular its viscosity, can be selected with regard to the physical properties of the substrate material, to ensure that the product can be absorbed quickly into and retained within the substrate, whilst at least a surface layer of the product can readily be flushed from the substrate by the liquid flow into which the product is dosed. Normally, the liquid product will be more viscous than the flowing liquid.

The operation of the device will now be explained. When the container 10 is initially installed on the device liquid product flows from the container 10 down through the delivery tube 34 into the volume 14 in cup 18. Initially the liquid escaping from the container 10 can be replaced by air, which enters the container via apertures 38 and the delivery tube 34. When the liquid in the well 14 covers the lower end 60 of the delivery tube 34, the passage of air to the container 10 is cut off. This in turn causes a drop in pressure in the free space 62 above the liquid in the container 10 as a small amount of liquid continues to flow into the cup 18. A state of equilibrium is rapidly reached in which the head of liquid in the well 14, which is exposed to atmospheric pressure via the vents 38, supports the liquid in the container 10, and the flow of liquid to the well 14 stops with the liquid level a little way above the mouth 60 of the delivery tube 34. If the level of liquid in the well 14 drops below the mouth 60 of the delivery tube 34, air can flow into the container 10 and the flow from the container 10 commences once more. In this way, the free liquid level in the well 14 is maintained substantially constant at or around the mouth 60 of the delivery tube 34.

The liquid product flows from the volume 14, through the notches 44 around the base of the tubular insert 24, into the circumferential gallery 42 at the base of the cup 18. From there, primarily under the influence of the hydrostatic forces, the fluid rises up the passages 46 between the cup 18 and the tubular insert 24 to a level corresponding to the liquid level in the well 14. The liquid is then dispersed from the cup 18. The liquid completes its upward passage to the underside of the substrate 6, through the upper, capillary portions of the passages 46 by virtue of capillary forces. From the upper end of the passages the liquid is then absorbed into the porous substrate 6, spreading through the area of the substrate 6.

The liquid product continues to flow from the volume 14 into the porous substrate 6, the volume 14 being topped up from the container 10 as necessary, until the substrate 6 becomes saturated. Once this happens, the substrate 6 ceases to draw liquid away from the top ends of the passages 46 and the capillary flow through the upper portions of the passages stops.

When the water closet is flushed, water flows down onto and over the platform 8 and the porous substrate 6 supported on it. The liquid product dose is flushed out of the substrate 6 by this flow of water and delivered to the water closet bowl 4. Once the flow of flushing water stops, and excess water has drained away, the substrate 6 is refilled with the liquid product by the process described above.

A small amount of water tends to be retained on the surface of the substrate. As this water evaporates it has been found to release the perfume component of the liquid product, providing a deodorising effect between flushes.

Unlike the prior art described above, since with the device of this invention there is no head of liquid acting from above the substrate 6, no additional liquid is absorbed once the substrate 6 is saturated. Consequently, the dose of liquid supplied can be relatively accurately and consistently controlled by selection of the properties of the porous substrate 6, in particular its absorbency and its physical dimensions.

It is preferable to inhibit the flow of flush water into the cup 18, as this will reduce the viscosity of liquid in the cup, and so affect the action dispersal mechanism, namely capillaries 46 and porous substrate 6. Thus connector 24 has an upper end 28 which forms a protective collar.

Various modifications can be made from the specifically described example without departing from the invention. For example, the platform 8 supporting the porous substrate 6 may be dispensed with, or more preferably lowered, if the substrate 6 is sufficiently rigid to support itself (or alternative support means are provided), the substrate 6 being secured between the flange on the tubular insert and the top of the cup. In this way, flushing water can be directed over both the upper and lower surfaces of the substrate 6 which forms the delivery plate per se.

FIGS. 4 to 8 show a second, preferred embodiment of the invention in which the porous substrate 6 of the first embodiment is replaced by capillary grooves formed in the upper surface of the platform 8 to form a delivery plate 80. Various modifications are made to the cup arrangement 18, in particular to enable the capillary passages 46 to deliver liquid 13 on to the upper surface of the plate 80.

Referring to FIG. 4, container 10 is shown only at its outlet region, but is closed at its upper part as for the embodiment of FIGS. 1 to 13. The cup 18′ is integrally formed with substantially flat delivery plate 80 forming an enlarged area adjacent the cup. A prong 61 extends up from the bottom wall 26′ of the cup 18′ to displace the seal 58 (see FIG. 2) of the closure 54′ of the container 10. Closure 54′ has inner and outer walls 63, 64 which are joined by a web 65 and embrace the neck 52 of the container 10 in a liquid tight seal. The closure 54′ has a connecting portion 66 which extends away from the container 10 to define a mouth 60. Seal 58 is held at lip 65′ on the inner rim of web 65, prior to being displaced by prong 61.

To form the capillary system for transporting liquid 13 from the inner volume 14 of cup 18, a grooved insert 70 is provided. It can be seen that insert 70 and connecting portion 66 perform a similar function to insert 24 of the embodiment of FIGS. 1 to 3.

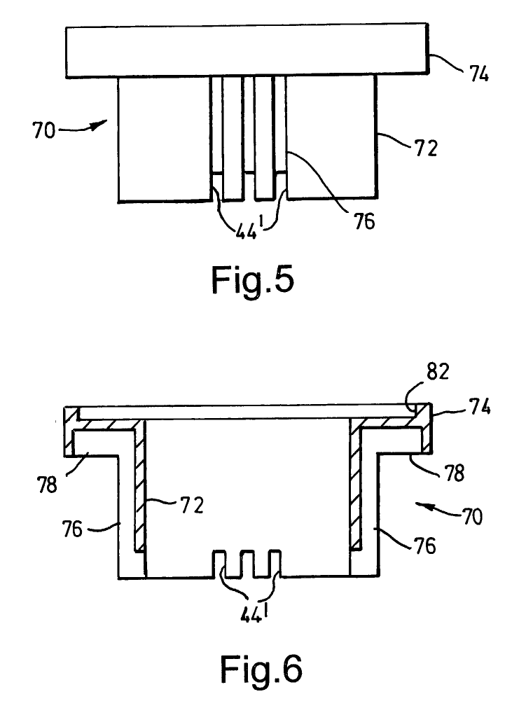

FIG. 5 shows a side view of insert 70 which comprises a circular cross-section cylinder wall 72 with a radially extending collar 74 at its upper end. Cylinder 72 is a snug fit in the cup 18.

Smooth walled capillary grooves 76 are formed in the outer surface of cylinder 72 around the complete periphery (only three are shown in FIGS. 5 and 6). At the lower end of each capillary 76, notches 44′ extend completely through the cylinder wall 72, similar to notches 44 in the embodiment of FIGS. 1 to 3.

FIG. 6 is a cross-section through the insert 70 on a diameter. It can be seen that capillaries 76 extend up into the collar 74 and across the underside 78. Thus, referring back to FIG. 4, the capillaries form a passage for liquid from volume 14, up between insert 70 and the wall of cup 18, to the upper surface 80′ of plate 80.

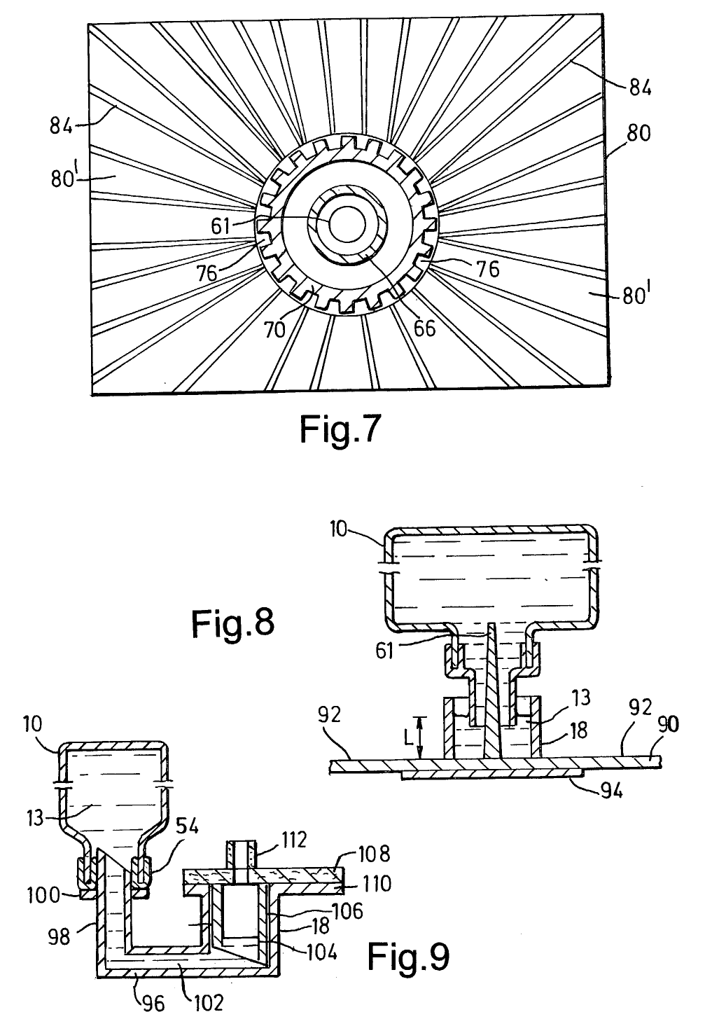

The upper surface of the collar 74 is recessed at 82 to provide an air gap around the cap 54′. It will be appreciated that a support or guide will also be provided to support container 10 in position and this may be in the form of a surrounding cage structure as known in the art. FIG. 7 shows the upper surface 80′ of the plate 80 with capillary grooves 84 in the surface 80′, extending away from the cup 18 and insert 70.

The operation of the device of FIGS. 4 to 7 is similar to the embodiment of FIGS. 1 to 3. Container 10 is inserted over the prong 61 to displace seal 58. Liquid 13 flows into volume 14, through notches 44′ and rises up capillaries 76. The liquid in volume 14 reaches a level L just above mouth 60′, when it is balanced by the partial vacuum created in container 10. However, the liquid in capillaries 76 will rise further, because of the capillary action, until it moves onto the surface 80 where it is conducted away in capillary channels 84.

As with the embodiment of FIGS. 1 to 3, it can be seen that the flow of liquid 13 on to plate 80 is substantially independent of the amount of liquid remaining in container 10, and is governed by the liquid level L in cup 18 and the capillary action generated by capillaries 76. As level L falls below mouth 60′, air can enter container 10 to allow more liquid to fall into volume 14.

The cross-section of the capillaries 76 can be modified to suit the viscosity of the liquid 13, a more viscous liquid requiring a larger capillary size as the rate of flow of the liquid through a given capillary is slower for a more viscous liquid. Also, the number of capillaries can be adjusted. It is desirable to ensure sufficient liquid flow to replenish the dosage delivered to porous member 6 or plate surface 80′ within about 30 seconds to 10 minutes.

An experiment was conducted to illustrate the effect of viscosity of the liquid on the vertical flow of liquid in a capillary.

Two glass plates were spaced apart from an amount “d” and dipped into a typical formulation shown in Table 1, adjusted for viscosity. The height reached by the liquid after 10 minutes was noted, and the results are shown in Table 2.

TABLE 1

| | | | Viscosity in cP |
|---|---|---|---|
| Formulation | Formulation Details | | Spindle 2 Speed |
| Reference | Wt. % | | 6 |
| LR126 “d” | Water (mains) | balance | 3400 |
| | Natrosol Cellulose Thickener | 0.4 | |
| | Preservative | 0.1 | |
| | Anionic Surfactant | 26.5 | |
| | Nonionic Surfactant | 5.0 | |
| | Solvents | 10.0 | |
| | Antioxidant | 0.004 | |
| | Perfume | 10.0 | |
| | Dye | 0.0024 | |
| RLR 067 | As LR126 “d” but; | 0.00 | 150 |
| | Natrosol | | |
| RLR069 | As LR126 “d” but; | 0.10 | 350 |
| | Natrosol | | |
| RLR070 | As LR126 “d” but; | 0.20 | 850 |
| | Natrosol | | |
| RLR071 | As LR126 “d” but; | 0.45 | 5100 |
| | Natrosol | | |
| RLR072 | As LR126 “d” but; | 0.30 | 1925 |
| | Natrosol | | |
| RLR074 | As LR126 “d” but; | 0.35 | 2500 |
| | Natrosol | | |

Viscosity measured in a Brookfield LV viscometer at 20° C., spindle 2 speed 6.

TABLE 2

| | Gap between the | |
|---|---|---|
| Viscosity/(centipoise) | plates/(mm) | Vertical height/(mm) |
| 150 | 1.25 | 2.3 |
| 150 | 1.0 | 3.5 |
| 150 | 0.75 | 6.0 |
| 150 | 0.5 | 8.5 |
| 150 | 0.25 | 16.0 |
| 350 | 1.25 | 2.5 |
| 350 | 1.0 | 5.0 |
| 350 | 0.75 | 6.0 |
| 350 | 0.5 | 9.0 |
| 350 | 0.25 | 16.0 |
| 850 | 1.25 | 2.5 |
| 850 | 1.0 | 3.0 |
| 850 | 0.75 | 6.5 |
| 850 | 0.5 | 8.0 |
| 850 | 0.25 | 16.0 |
| 1925 | 1.25 | 3.0 |
| 1925 | 1.0 | 4.5 |
| 1925 | 0.75 | 6.0 |
| 1925 | 0.5 | 9.0 |
| 1925 | 0.25 | 14.0 |
| 2500 | 1.25 | 2.5 |
| 2500 | 1.0 | 4.0 |
| 2500 | 0.75 | 6.0 |
| 2500 | 0.5 | 8.5 |
| 2500 | 0.25 | 12.0 |
| 3400 | 1.25 | 2.5 |
| 3400 | 1.0 | 4.5 |
| 3400 | 0.75 | 5.5 |
| 3400 | 0.5 | 8.5 |
| 3400 | 0.25 | 11.0 |
| 5100 | 1.25 | 3.2 |
| 5100 | 1.0 | 4.0 |
| 5100 | 0.75 | 5.0 |
| 5100 | 0.5 | 8.5 |
| 5100 | 0.25 | 11.0 |

It will be appreciated that the insert 70 of the FIGS. 4 to 7 embodiment could be used to deliver liquid to a porous member, such as members 6 of FIGS. 1 to 3.

Also, in place of a capillary system for delivering liquid upwards from, cup 18, liquid could be dispensed from the bottom of the cup.

Thus, FIG. 8 shows schematically a system in which the bottom wall 26 of the cup 18 is formed by a porous plate 90 which extends beyond the cup 18 to form a delivery plate. Liquid will be drawn from the cup 18 into the exposed region 92 of the plate, where flush water can wash liquid from the plate. A non-porous cover 94 is provided below the region of the cup 18 to prevent liquid dripping straight through the plate 90. The head L of liquid above the plate 90 will be substantially constant.

In the embodiment of FIG. 9, The cup 18 is connected to the container 10 by a conduit 96. The container is inverted over a vertical arm 98 of the conduit 96, resting on a flange 100. Liquid flows into the cup 18 until the horizontal passage 102 of the conduit is filled, preventing air returning into the container 10. An insert 104 forms a capillary passage 106 with the wall of the cup 18, to conduct liquid 13 up to a porous substrate 108 which is supported at the mouth of the cup 18 on a plate 110. The volume 14 of cup 18 is open to atmosphere through a tube 112, which also serves to inhibit the flow of flush water into the cup 18. It will be appreciated that insert 70 could also be used with this embodiment to deliver liquid on to the top of porous plate 108 or a grooved plate.

Various modifications will be apparent to those in the art and it is desired to include all such modifications as fall within the scope of the accompanying claims. For example, the porous substrate may be a self supporting plate, preferably substantially flat and providing an enlarged area adjacent the cup for dispersal of the liquid. In place of the capillaries and porous substrate or grooved delivery plate another wicking system may be used to disperse the liquid from the cup.

What is claimed is

1. A liquid delivery device comprising a container for the liquid, the container having an outlet which, in use, is below the upper level of liquid in the container, and a cup surrounding a mouth of the container outlet, the cup being open to the atmosphere, whereby in use liquid flows into the cup until the mouth is covered by liquid in the cup, and wherein dispersal means is provided to disperse liquid from the cup.

2. A liquid delivery device as claimed in claim 1, wherein the dispersal means includes a porous member in liquid communication with the cup.

3. A liquid delivery device as claimed in claim 1, wherein the dispersal means includes a plate having capillary channels formed in a surface thereof, the capillary channels being in liquid communication with the cup.

4. A liquid delivery device as claimed in claim 1, wherein the dispersal means includes a capillary for transporting the liquid upwards from the level of liquid in the cup.

5. A liquid delivery device as claimed in claim 4, wherein the capillary is formed between an inner wall of the cup and an insert member inserted in the cup.

6. A liquid delivery device as claimed in claim 5, wherein the insert member has a plurality of capillary channels formed in an outer wall thereof.
